(19) United States
(12) Patent Application Publication
(10) Pub. No.: US 2009/0062839 A1
(43) Pub. Date: Mar. 5, 2009

(54) BARBED STENT VASCULAR OCCLUSION DEVICE

(75) Inventor: Michael R. Kurrus, Ellettsville, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/848,782

(22) Filed: Aug. 31, 2007

(51) Int. Cl. A61M 29/00

(52) U.S. Cl. 606/198

(57) ABSTRACT

A vascular occlusion device for occluding a body cavity. The device includes a tubular scaffold extending from a proximal end to a distal end. The scaffold is formed from a plurality of interconnected and articulated members configured to self-expand into an open configuration. A plurality of barbs extend from the articulated members, each barb including an anchoring end. The anchoring end is disposed radially outward from the scaffold in the open configuration and adapted to embed into the cavity walls. A radially expandable substance is disposed within a device lumen. The substance is configured to promote body tissue growth within the body cavity to occlude the body cavity. In one example, the body cavity includes a patent foramen ovale.

### BACKGROUND OF THE INVENTION

1. Field of the Invention

The present invention generally relates to vascular occlusion devices. More specifically, the invention relates to a vascular occlusion device for repairing an atrial septal defect.

2. Description of Related Art

A number of different devices may be used to occlude a body cavity including, for example, a blood vessel. When it is desirable to quickly occlude a blood vessel, an inflatable balloon may be used. However, balloon's have the disadvantage of being temporary. Another example of an occlusion device includes embolization coils. Embolization coils are permanent and promote blood clots or tissue growth over a period of time, thereby occluding the body cavity. However, while the blood clots or the tissue grows, blood may continue to flow past the coil and through the body cavity. It may take a significant period of time for sufficient tissue to grow to fully occlude the body cavity. This leaves a patient open to a risk of injury from the condition which requires the body cavity be occluded. The condition may include, but is not limited to, a patent foramen ovale.

In view of the above, it is apparent that there exists a need for an improved vascular occlusion device.

### SUMMARY OF THE INVENTION

In satisfying the above need, as well as overcoming the enumerated drawbacks and other limitations of the related art, the present invention provides a vascular occlusion device for occluding a body cavity. The device includes a tubular scaffold extending from a proximal end to a distal end. The scaffold is formed from a plurality of interconnected and articulated members configured to self-expand into an open configuration. A plurality of barbs extend from the articulated members to an anchoring end. The anchoring end is disposed radially outward from the scaffold in the open configuration and adapted to embed into the cavity walls. A radially expandable substance is disposed within a device lumen. The substance is configured to promote body tissue growth from body cavity walls to occlude the body cavity. In some examples, the anchoring ends of the barbs are disposed substantially flush along the scaffold in a closed configuration.

The tubular scaffold may be any of various self-expanding stents. In a first embodiment, the tubular wall further comprises at least one self-expanding ring structure, the ring structure being formed from the plurality of articulated members. For example, each articulated member of the ring structure may have a proximal tip and a distal tip. Each of the proximal and distal tips are attached at a joint to a respective proximal or distal tip of an adjacent member to form the ring structure.

In one example of this embodiment, a plurality of the ring structures are coaxially aligned from the proximal to the distal end of the device. Each of the ring structures are attached to at least one adjacent ring structure. In another example, the ring structures may be attached together by a plurality of longitudinal members. In yet another example, the articulated members and joints of the ring structures form a sinusoidal pattern.

In a second embodiment, the radially expandable substance may include an extracellular matrix, polyester, rayon, nylon, polytetrafluoroethylene, biocompatible polyurethanes, and mixtures thereof. In some examples, the extracellular matrix includes small intestine submucosa (SIS). In other examples, the SIS is compressed for passage through a lumen of a sheath and is expanded when disposed outside of the lumen. In other examples, the radially expandable substance forms an interconnected matrix of fibers within the device lumen in the open configuration.

In a third embodiment, the tubular scaffold barbs are made of a shape memory material. The shape memory material may include, for example, alloys of nickel-titanium (Nitinol).

The present invention also provides a vascular occlusion assembly. The assembly includes a delivery apparatus including an outer sheath having a proximal part extending to a distal part and defining a sheath lumen. An inner elongate element is disposed within the sheath lumen and has a proximal segment extending to a distal segment. The outer sheath is configured to translate axially relative to the inner element. Any of the embodiments of the occlusion device described above may be disposed within the sheath lumen in engagement with the distal segment of the inner element.

The occlusion device is coaxially arranged within the sheath lumen in the closed configuration such that the radially expandable substance is compressed within the device lumen. The occlusion device is deployable through the distal part of the outer sheath by means of relative axial movement of the outer sheath. The scaffold, barbs, and extracellular matrix self-expand into the open configuration after deployment of the occlusion device.

The present invention additionally provides a method of occluding a body cavity. The method includes providing any of the above occlusion devices within the body cavity, positioning the occlusion device within the body cavity to promote body tissue growth, expanding the occlusion device within the body cavity, and attaching the anchoring ends of occlusion device to the body walls of the body cavity. In some embodiments, the body cavity may be a heart having an atrial septal defect. The atrial septal defect may include, for example, a patent foramen ovale of a heart.

Further objects, features and advantages of this invention will become readily apparent to persons skilled in the art after a review of the following description, with reference to the drawings and claims that are appended to and form a part of this specification.

### BRIEF DESCRIPTION OF THE DRAWINGS

FIG. 1A is a side view of a device for occluding a body cavity;

FIG. 1B is an end view of the device of FIG. 1;

FIG. 2 is a partial sectional view of a delivery apparatus incorporating the device of FIG. 1A;

FIG. 3A is a plan view of a catheter assembly for introducing the device of FIG. 1 into the body cavity;

FIG. 3B is an exploded view of the components of the assembly of FIG. 3A;

FIG. 4A is a section view of a human heart showing the assembly of FIG. 3A introducing the device of FIG. 1 into a patent foramen ovale;

FIG. 4B is a detail view showing the device of FIG. 1 in position within the patent foramen ovale; and

FIG. 5 is a flow chart illustrating a method of occluding a body cavity.

### DETAILED DESCRIPTION

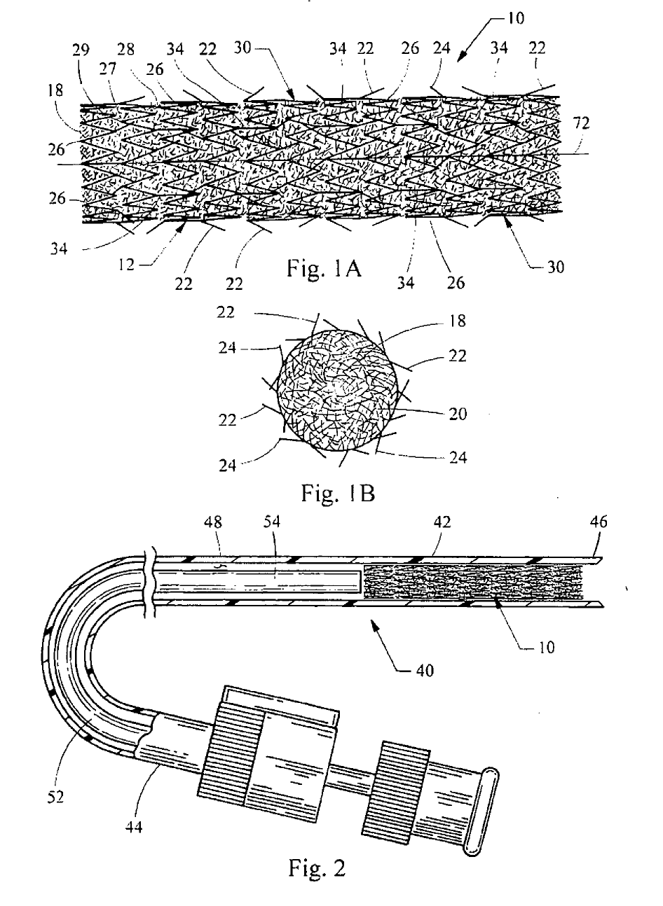

Referring now to FIGS. 1A and 1B, an occlusion device embodying the principles of the present invention is illustrated therein and designated at 10. As its primary components, the occlusion device 10 includes tubular scaffold 12 extending from a proximal end 14 to a distal end 16 and defining a device lumen 18 therethrough. A plurality of barbs 22 are attached to the scaffold 12, and a radially expandable substance 20 is at least partially disposed within the device lumen 18 and attached to the scaffold 12. The substance 20 is configured to promote tissue growth within a body cavity.

In one embodiment, the scaffold 12 is formed from a plurality of interconnected and articulated members 26 configured to expand into an open configuration as best shown in FIGS. 1A and 1B. Each of the articulated members 26 have a proximal tip 27 and a distal tip 28 with each of the proximal and distal tips 27 and 28 being attached at a joint 29 to a respective proximal or distal tip 27 or 28 of an adjacent member 26. In the example shown, the articulated members 26 are arranged to form a self-expanding ring shaped structure 30.

A plurality of the ring structures 30 may be, for example, coaxially aligned from the proximal end 14 to the distal end 16 of the device 10 along a longitudinal axis 32. In this embodiment, each of the ring structures 30 are attached to at least one adjacent ring structure. In some examples, the ring structures 30 may be attached together at the joints 29 (not shown). In other examples, the ring structures 30 may be attached together by a plurality of longitudinal member 34 as shown in FIG. 1A. In yet another example, the articulated members 26 and joints 29 may be configured to form a sinusoidal pattern.

While the above description illustrates one exemplary embodiment of the tubular scaffold 12, it should be understood that the scaffold 12 may include any of a variety of self-expanding devices such as, for example, stents. Some examples of self-expanding stents include, but are not limited to, those disclosed in U.S. Pat. No. 4,580,568; U.S. Pat. No. 5,035,706; U.S. Pat. No. 5,507,767; and U.S. Pat. No. 6,042,606 all of which are incorporated herein by reference.

The barbs 22 extend from the articulated members 26 and include an anchoring end 24. The barbs may, if applicable, extend from the longitudinal members 34 (not shown). As best shown in FIG. 1B, the anchoring end 24 is disposed radially outward from the scaffold 12 and longitudinal axis 32 in the open configuration shown in FIGS. 1A and 1B. The barbs 22 may, for example, be separately attached to, or formed integrally with, the articulated members 26. The anchoring end 24 is adapted to be embedded into walls of the body cavity in order to hold the device 10 in place and prevent migration once deployed within the body cavity. One example of the barb 22 includes, but is not limited to, those disclosed in U.S. Pat. No. 7,081,132 which is incorporated herein by reference.

Turning now to FIG. 2, the device 10 is shown disposed within a delivery apparatus 40 including an outer sheath 42 having a proximal part 44 and a distal part 46 and defining a sheath lumen 48. An inner elongate element 50 is disposed within the sheath lumen 48 and has a proximal segment 52 extending to a distal segment 54. The outer sheath 42 is configured to translate axially relative to the inner element 50. An occlusion device 10 is disposed within the sheath lumen 48 in releasable engagement with the distal segment 54 of the inner element 50. As shown, the scaffold 12 has a closed configuration when disposed within the sheath lumen 48. In one example of the closed configuration, the barbs 22 are disposed substantially flush along the scaffold 12 to permit the device 10 to translate axially through the sheath lumen 48 with damaging the outer sheath 42. The device 10 is deployable through the distal part of the outer sheath 42 by relative axial movement of the outer sheath 42 to the inner element 50. Upon deployment of the device 10, the scaffold 12, barbs 22 and expandable substance 20 self-expand into the open configuration (see FIGS. 1A and 1B).

The radially expandable substance 20 of the device 10, being coaxially arranged within the sheath lumen 48, is compressed within the device lumen 18. While most of the substance 20 is disposed within the device lumen 18 in both the open and closed configurations, in some examples it is possible for a portion of the substance 20 to protrude beyond the scaffold 12 and remain within the scope of the present invention.

The radially expandable substance 20 may be any suitable compressible and expandable material for promoting tissue growth within a body cavity. This includes, for example an extracellular matrix (ECM), polyester, rayon, nylon, polytetrafluoroethylene, biocompatible polyurethanes, and combinations thereof. In some examples, the radially expandable substance forms an interconnected matrix or lattice of fibers within the device lumen 18 when expanded into the open configuration.

As known, ECM is a complex structural entity surrounding and supporting cells found within tissues. More specifically, ECM includes structural proteins (for example, collagen and elastin), specialized protein (for example, fibrillin, fibronectin, and laminin), and proteoglycans, a protein core to which are attached long chains of repeating disaccharide units termed glycosaminoglycans.

In a preferred embodiment, the extracellular matrix is comprised of small intestinal submucosa (SIS). As known, SIS is a resorbable, acellular, naturally occurring tissue matrix composed of ECM proteins and various growth factors. SIS is derived from the porcine jejunum and functions as a remodeling bioscaffold for tissue repair. SIS has characteristics of an ideal tissue engineered biomaterial and can act as a bioscaffold for remodeling of many body tissues including skin, body wall, musculoskeletal structure, urinary bladder, and also supports new blood vessel growth. SIS may be used to induce site-specific remodeling of both organs and tissues depending on the site of implantation. In practice, host cells are stimulated to proliferate and differentiate into site-specific connective tissue structures, which have been shown to completely replace the SIS material in time.

In this embodiment, SIS is used to adhere to walls of a body cavity in which the device 10 is deployed and to promote body tissue growth within the body cavity. SIS has a natural adherence or wetability to body fluids and connective cells comprising the connective tissue of the walls of a body cavity. Since the device 10 is intended to permanently occlude the body cavity, the device 10 is positioned such that host cells of the wall will adhere to the SIS and subsequently differentiate, growing into the SIS and eventually occluding the body cavity with the tissue of the walls to which the substance 20 was originally adhered.

One example of the biocompatible polyurethane is sold under the trade name THORALON (THORATEC, Pleasanton, Calif.). Descriptions of suitable biocompatible polyureaurethanes are described in U.S. Pat. Application Publication No. 2002/0065552 A1 and U.S. Pat. No. 4,675,361, both of which are herein incorporated by reference. Briefly, these publications describe a polyurethane base polymer (referred to as BPS-215) blended with a siloxane containing surface modifying additive (referred to as SMA-300). Base polymers containing urea linkages can also be used. The concentration of the surface modifying additive may be in the range of 0.5% to 5% by weight of the base polymer.

The SMA-300 component (THORATEC) is a polyurethane comprising polydimethylsiloxane as a soft segment and the reaction product of diphenylmethane diisocyanate (MDI) and 1,4-butanediol as a hard segment. A process for synthesizing SMA-300 is described, for example, in U.S. Pat. Nos. 4,861,830 and 4,675,361, which are incorporated herein by reference.

The BPS-215 component (THORATEC) is a segmented polyetherurethane urea containing a soft segment and a hard segment. The soft segment is made of polytetramethylene oxide (PTMO), and the hard segment is made from the reaction of 4,4′-diphenylmethane diisocyanate (MDI) and ethylene diamine (ED).

THORALON can be manipulated to provide either porous or non-porous structures. The present invention envisions the use of non-porous THORALON. Non-porous THORALON can be formed by mixing the polyetherurethane urea (BPS-215) and the surface modifying additive (SMA-300) in a solvent, such as dimethyl formamide (DMF), tetrahydrofuran (TH F), dimethyacetamide (DMAC), dimethyl sulfoxide (DMSO). The composition can contain from about 5 wt % to about 40 wt % polymer, and different levels of polymer within the range can be used to fine tune the viscosity needed for a given process. The composition can contain less than 5 wt % polymer for some spray application embodiments. The entire composition can be cast as a sheet, or coated onto an article such as a mandrel or a mold. In one example, the composition can be dried to remove the solvent.

THORALON has been used in certain vascular applications and is characterized by thromboresistance, high tensile strength, low water absorption, low critical surface tension, and good flex life. THORALON is believed to be biostable and to be useful in vivo in long term blood contacting applications requiring biostability and leak resistance. Because of its flexibility, THORALON is useful in larger vessels, such as the abdominal aorta, where elasticity and compliance is beneficial.

A variety of other biocompatible polyurethanes/polycarbamates and urea linkages (hereinafter “—C(O)N or CON type polymers”) may also be employed. These include CON type polymers that preferably include a soft segment and a hard segment. The segments can be combined as copolymers or as blends. For example, CON type polymers with soft segments such as PTMO, polyethylene oxide, polypropylene oxide, polycarbonate, polyolefin, polysiloxane (i.e. polydimethylsiloxane), and other polyether soft segments made from higher homologous series of diols may be used. Mixtures of any of the soft segments may also be used. The soft segments also may have either alcohol end groups or amine end groups. The molecular weight of the soft segments may vary from about 500 to about 5,000 g/mole.

Preferably, the hard segment is formed from a diisocyanate and diamine. The diisocyanate may be represented by the formula OCN—R—NCO, where —R— may be aliphatic, aromatic, cycloaliphatic or a mixture of aliphatic and aromatic moieties. Examples of diisocyanates include MDI, tetramethylene diisocyanate, hexamethylene diisocyanate, trimethyhexamethylene diisocyanate, tetramethylxylylene diisocyanate, 4,4′-dicyclohexylmethane diisocyanate, dimer acid diisocyanate, isophorone diisocyanate, metaxylene diisocyanate, diethylbenzene diisocyanate, decamethylene 1,10 diisocyanate, cyclohexylene 1,2-diisocyanate, 2,4-toluene diisocyanate, 2,6-toluene diisocyanate, xylene diisocyanate, m-phenylene diisocyanate, hexahydrotolylene diisocyanate (and isomers), naphthylene-1,5-diisocyanate, 1-methoxyphenyl 2,4-diisocyanate, 4,4′-biphenylene diisocyanate, 3,3′-dimethoxy-4,4′-biphenyl diisocyanate and mixtures thereof.

The diamine used as a component of the hard segment includes aliphatic amines, aromatic amines and amines containing both aliphatic and aromatic moieties. For example, diamines include ethylene diamine, propane diamines, butanediamines, hexanediamines, pentane diamines, heptane diamines, octane diamines, m-xylylene diamine, 1,4-cyclohexane diamine, 2-methypentamethylene diamine, 4,4′-methylene dianiline, and mixtures thereof. The amines may also contain oxygen and/or halogen atoms in their structures.

Other applicable biocompatible polyurethanes include those using a polyol as a component of the hard segment. Polyols may be aliphatic, aromatic, cycloaliphatic or may contain a mixture of aliphatic and aromatic moieties. For example, the polyol may be ethylene glycol, diethylene glycol, triethylene glycol, 1,4-butanediol, 1,6-hexanediol, 1,8-octanediol, propylene glycols, 2,3-butylene glycol, dipropylene glycol, dibutylene glycol, glycerol, or mixtures thereof.

Biocompatible CON type polymers modified with cationic, anionic and aliphatic side chains may also be used. See, for example, U.S. Pat. No. 5,017,664. Other biocompatible CON type polymers include: segmented polyurethanes, such as BIOSPAN; polycarbonate urethanes, such as BIONATE; and polyetherurethanes, such as ELASTHANE; (all available from POLYMER TECHNOLOGY GROUP, Berkeley, Calif.).

Other biocompatible CON type polymers can include polyurethanes having siloxane segments, also referred to as a siloxane-polyurethane. Examples of polyurethanes containing siloxane segments include polyether siloxane-polyurethanes, polycarbonate siloxane-polyurethanes, and siloxane-polyurethane ureas. Specifically, examples of siloxane-polyurethane include polymers such as ELAST-EON 2 and ELAST-EON 3 (AORTECH BIOMATERIALS, Victoria, Australia); polytetramethyleneoxide (PTMO) and polydimethylsiloxane (PDMS) polyether-based aromatic siloxane-polyurethanes such as PURSIL-10, -20, and -40 TSPU; PTMO and PDMS polyether-based aliphatic siloxane-polyurethanes such as PURSIL AL-5 and AL-10 TSPU; aliphatic, hydroxy-terminated polycarbonate and PDMS polycarbonate-based siloxane-polyurethanes such as CARBOSIL-10, -20, and -40 TSPU (all available from POLYMER TECHNOLOGY GROUP). The PURSIL, PURSIL-AL, and CARBOSIL polymers are thermoplastic elastomer urethane copolymers containing siloxane in the soft segment, and the percent siloxane in the copolymer is referred to in the grade name. For example, PURSIL-10 contains 10% siloxane. These polymers are synthesized through a multi-step bulk synthesis in which PDMS is incorporated into the polymer soft segment with PTMO (PURSIL) or an aliphatic hydroxy-terminated polycarbonate (CARBOSIL). The hard segment consists of the reaction product of an aromatic diisocyanate, MDI, with a low molecular weight glycol chain extender. In the case of PURSIL-AL the hard segment is synthesized from an aliphatic diisocyanate. The polymer chains are then terminated with a siloxane or other surface modifying end group. Siloxane-polyurethanes typically have a relatively low glass transition temperature, which provides for polymeric materials having increased flexibility relative to many conventional materials. In addition, the siloxane-polyurethane can exhibit high hydrolytic and oxidative stability, including improved resistance to environmental stress cracking. Examples of siloxane-polyurethanes are disclosed in U.S. Pat. Application Publication No. 2002/0187288 A1, which is incorporated herein by reference.

In addition, any of these biocompatible CON type polymers may be end-capped with surface active end groups, such as, for example, polydimethylsiloxane, fluoropolymers, polyolefin, polyethylene oxide, or other suitable groups. See, for example the surface active end groups disclosed in U.S. Pat. No. 5,589,563, which is incorporated herein by reference.

At least part of the scaffold 12 and the barbs 22 of the device 10 may be made of any suitable material, for example, a superelastic material, stainless steel wire, cobalt-chromium-nickel-molybdenum-iron alloy, cobalt-chrome alloy, or stress relieved metal (e.g. platinum). It is understood that the scaffold 12 and barbs 22 may preferably be formed of any appropriate material that will result in a self-expanding device 10 capable of being percutaneously inserted and deployed within a body cavity, such as shape memory material. Shape memory materials or alloys have the desirable property of becoming rigid, i.e., returning to a remembered state, when heated above a transition temperature. A shape memory alloy suitable for the present invention is Ni—Ti available under the more commonly known name Nitinol. When this material is heated above the transition temperature, the material undergoes a phase transformation from martensite to austenite, such that the material returns to its remembered state. The transition temperature is dependent on the relative proportions of the alloying elements Ni and Ti and the optional inclusion of alloying additives.

In one embodiment, the scaffold 12 is made from Nitinol with a transition temperature that is slightly below a normal body temperature of humans, which is about 98.6° F. Thus, when the device 10 is deployed in a body vessel and exposed to normal body temperature, the alloy of the device 10 will transform to austenite, that is the remembered state. The remembered state includes the open configuration with the barbs 22 extending radially outward when the device 10 is deployed in the body cavity. If it is ever necessary to remove the device 10 from the body cavity, the device 10 is cooled to transform the material to martensite which is more ductile than austenite, making the device 10 more malleable. As a result, the device 10 can be more easily collapsed and pulled into a lumen of a catheter for removal.

In another embodiment, the device 10 is made from Nitinol with a transition temperature that is above normal body temperature of humans, which is about 98.6° F. Thus, when the device 10 is deployed in a body vessel and exposed to normal body temperature, the device 10 is in the martensitic state so that the device 10 is sufficiently ductile to bend or form into a desired shape. In the event it ever becomes necessary to remove the device 10, the device 10 is heated to transform the alloy to austenite so that the device 10 becomes rigid and returns to a remembered state, which for the device 10 is the closed configuration, for example, that shown in FIG. 2.

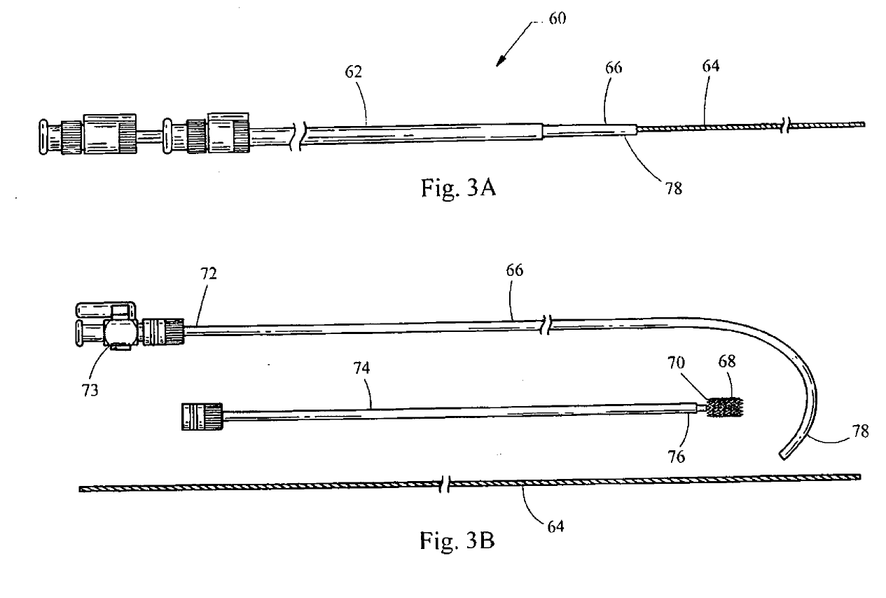

FIGS. 3A and 3B depict a delivery assembly 60 for introducing and retrieving an occlusion device 68 for occluding a body cavity in accordance with another embodiment of the present invention. As shown, the delivery assembly 60 includes a polytetrafluoroethylene (PTFE) introducer sheath 62 for percutaneously introducing an outer sheath 66 into a body vessel. Of course, any other suitable material for the introducer sheath 62 may be used without falling beyond the scope or spirit of the present invention. The introducer sheath 62 may have any suitable size, for example, between about three-french to eight-french. The introducer sheath 62 serves to allow the outer sheath 66 and an inner element 74 to be percutaneously inserted to a desired location in a body cavity through the body vessel. It should be understood that the inner element 74 includes catheters and other elongate pushing members including, for example, a stylet. The introducer sheath 62 receives the outer sheath 66 and provides stability to the outer sheath 66 at a desired entry location of the body vessel. For example, the introducer sheath 62 is held stationary within a common visceral artery, and adds stability to the outer sheath 66 as it is advanced through the introducer sheath 62 to an occlusion area in the body cavity.

As shown, the assembly 60 may also include a wire guide 64 configured to be percutaneously inserted within the body vessel to guide the outer sheath 66 to the occlusion area. The wire guide 64, which may be disposed through the center of the occlusion device, provides the outer sheath 66 with a path to follow as it is advanced within the body vessel. The size of the wire guide 64 is based on the inside diameter of the outer sheath 66 and the diameter of the body vessels that must be traversed to reach the desired body cavity.

When a distal portion 78 of the outer sheath 66 is at the desired location in the body cavity, the wire guide 64 is removed and the occlusion device 68, having a proximal end 70 releasably engaged with a distal segment 76 of the inner element 74, is inserted into the outer sheath 66. It should be noted that the occlusion device 68 may be any of the occlusion devices described above. The inner element 74 is advanced through the outer sheath 66 for deployment of the occlusion device 68 through the distal portion 78 to occlude, for example, a patent foramen ovale in a human heart.

As shown, the outer sheath 66 also has a proximal portion 72 including a hub 73 to receive the occlusion device 68 and the inner element 74 to be advanced therethrough. When the occlusion device 68 is inside of the outer sheath 66 the occlusion device 68 takes a radially compressed or closed configuration. The size of the outer sheath 66 is based on the size of the body vessel in which it percutaneously inserts, and the size of the occlusion device 68.

In the present embodiment, the occlusion device 68 and inner element 74 are coaxially disposed through the outer sheath 66, following removal of the wire guide 64, in order to position the occlusion device 68 to occlude, for example, the patent foramen ovale. The occlusion device 68 is guided through the outer sheath 66 by the inner element 74, preferably from the hub 72, and exits from the distal portion 78 of the outer sheath 66 at a location within the heart where occlusion of the patent foramen oval is desired.

The occlusion device 68 may be retrieved, should it ever become necessary. In one example, retrieval may be accomplished by positioning the distal portion 78 of the outer sheath 66 adjacent the deployed occlusion device 68 in the body cavity. The inner element 74 is advanced through the outer sheath 66 until the distal segment 76 of the inner element 74 protrudes from the distal portion 78 of the outer sheath 66. The distal segment 76 is coupled to the proximal portion 70 of the occlusion device 68. After the occlusion device 68 has been freed from walls of the body cavity, the inner element 74 is retracted proximally, drawing the occlusion device 68 into the outer sheath 66. Other methods may be implemented without falling beyond the scope or spirit of the present invention.

It is understood that the assembly described above is merely one example of an assembly that may be used to deploy the device in a body vessel. Of course, other apparatus, assemblies and systems may be used to deploy any embodiment of the device without falling beyond the scope or spirit of the present invention.

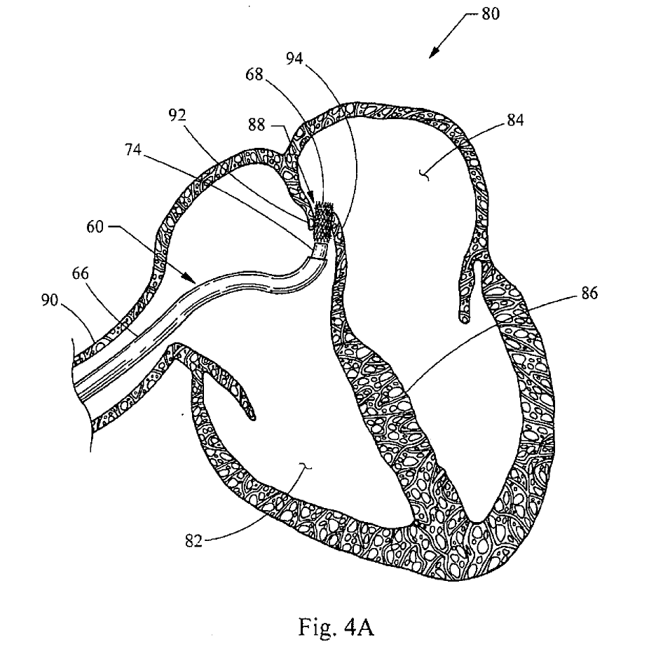

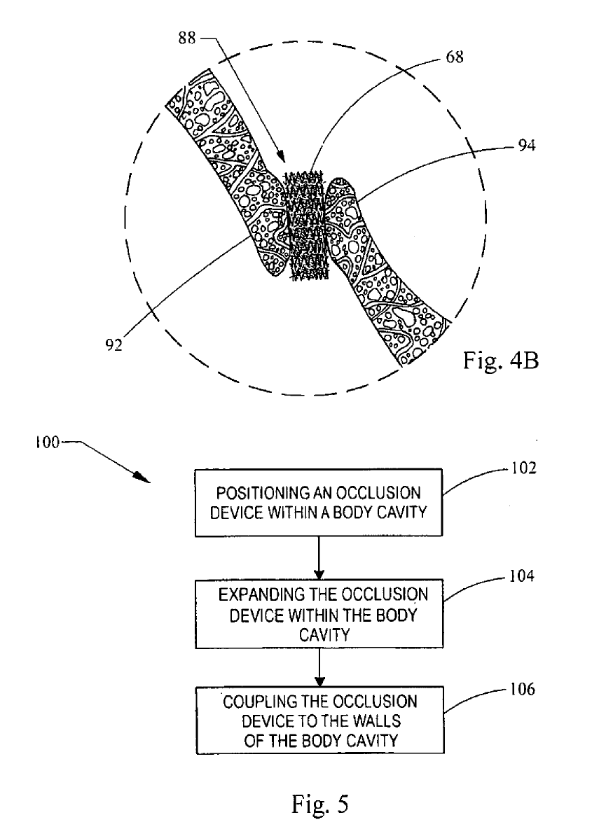

As mentioned above, one exemplary application of the delivery assembly 60 may be to treat a patent foramen ovale in a human heart 80 as shown in FIGS. 4A and 4B. It should be noted that this is merely one example and the delivery assembly 60 may be used in a variety of other applications to occlude various other body cavities without departing from the scope or spirit of the present invention. FIG. 4A shows a sectional view of a human heart 80 having a right atrium 82 and a left atrium 84. An atrial septum 86 divides the right atrium 82 from the left atrium 84 and includes a patent foramen oval 88. The patent foramen oval 88 is an opening in the atrial septum 86 that allows blood in the right and left atria 82 and 84 to fluidly communicate therebetween.

In a fetus, a foramen ovale is a natural hole in the atrial septum 88 that allows blood to bypass the fetus' lungs when in a mother's womb since the fetus relies on the mother to provide oxygen through the umbilical cord. At birth the foramen ovale normally closes when increased blood pressure in the left atrium forces the opening to close. Overt time tissue growth closes the opening permanently. However, in some people the opening does not close permanently, in which case the opening is called a patent foramen ovale.

As shown in FIGS. 4A and 4B, the patent foramen ovale 88 acts like a flap valve, having a right flap 92 and a left flap 94, between the two atria 82 and 84. Normally, higher pressure in the left atrium 84 keeps the flaps closed. However, during certain conditions, such as when there is increased pressure inside the chest around the heart, the flaps may open and blood may travel from the right atrium 82 to the left atrium 84. If a clot is present in the right atrium 82 it can, for example, enter the left atrium 84 and travel from there to the brain (causing a stroke) or into a coronary artery (causing a heart attack).

Therefore, it is desirable to close the patent foramen ovale 88 permanently. Turning to FIG. 4A, the delivery assembly 60 may be percutaneously introduced into a body vessel 90 and directed into, for example, the right atrium 82 and maneuvered adjacent the patent foramen ovale 88. The outer sheath 66 is retracted proximally from the occlusion device 68. The inner element 74 may be used to position the occlusion device 68 within the patent foramen ovale 88 such that, for example, small intestine submucosa (SIS) disposed within the occlusion device 68 is positioned between the right and left flaps 92 and 94. As best shown in FIG. 4B, the occlusion device 68 is positioned between and in contact with each of the flaps 92 and 94. Barbs radially extend from the occlusion device 68 and secure the device 68 in place. In some embodiments additional securing means may also be used including, for example, sutures. As a result, the flaps 92 and 94 of the patent foramen ovale 88 are held in contact with the occlusion device 68 and, as described above, body tissue of the atrial septum 86 will quickly differentiate and grow to completely replace the SIS material, thereby permanently closing the patent foramen ovale 88.

FIG. 5 is a flow chart illustrating a method 100 of occluding a body cavity. The method 100 includes at box 102 positioning any of the above described occlusions devices within a body cavity. Box 104 includes expanding the occlusion device within the body cavity and box 106 includes coupling the occlusion device to the walls of the body cavity.

As a person skilled in the art will readily appreciate, the above description is meant as an illustration implementing the principles this invention. This description is not intended to limit the scope or application of this invention in that the invention is susceptible to modification, variation and change, without departing from the spirit of this invention, as defined in the following claims.

We claim:

1. A vascular occlusion device for occluding a body cavity defined by cavity walls, the device comprising:
a tubular scaffold extending from a proximal end to a distal end and defining a device lumen therethrough, the scaffold being formed from a plurality of interconnected and articulated members configured to self-expand into an open configuration, a plurality of barbs extend from the articulated members with each barb including an anchoring end, the anchoring end being disposed radially outward from the scaffold in the open configuration and adapted to embed into the cavity walls; and
a radially expandable substance being disposed within the device lumen and attached to at least one of the articulated members, the substance being configured to promote body tissue growth within the body cavity to occlude the body cavity.

2. The device of claim 1 wherein the tubular scaffold further comprises a closed configuration wherein the anchoring end of the barbs are disposed substantially flush along the scaffold.

3. The device of claim 1 wherein the tubular scaffold further comprises at least one self-expanding ring structure, the ring structure being formed from the plurality of articulated members.

4. The device of claim 3 wherein each articulated member has a proximal tip and a distal tip and each of the proximal and distal tips are attached at a joint to a respective proximal or distal tip of an adjacent member to form the ring structure.

5. The device of claim 3 wherein the tubular scaffold further comprises a plurality of the ring structures being coaxially aligned from the proximal to the distal end of the device, each of the ring structures being attached to at least one adjacent ring structure.

6. The device of claim 5 wherein the ring structures are attached together by a plurality of longitudinal members.

7. The device of claim 6 wherein the articulated members and joints of the ring structures form a sinusoidal pattern.

8. The device of claim 1 wherein the substance further comprises at least one of an extracellular matrix, polyester, rayon, nylon, polytetrafluoroethylene, biocompatible polyurethanes, and mixtures thereof.

9. The device of claim 8 wherein the extracellular matrix further comprises small intestine submucosa.

10. The device of claim 9 wherein the small intestine submucosa is compressed for passage through a lumen of a sheath and is expanded when the device is disposed outside of the lumen of the sheath.

11. The device of claim 1 wherein the radially expandable substance forms an interconnected matrix of fibers within the device lumen in the open configuration.

12. The device of claim 1 wherein the tubular scaffold and barbs are made of a shape memory material.

13. The device of claim 12 wherein the shape memory material includes alloys of nickel-titanium.

14. A vascular occlusion assembly for occluding a body cavity defined by cavity walls, the assembly comprising:
a delivery apparatus including an outer sheath having a proximal part extending to a distal part and defining a sheath lumen therein, an inner elongate element being disposed within the sheath lumen and having a proximal segment extending to a distal segment, the outer sheath being configured to translate axially relative to the inner element; an occlusion device being disposed within the sheath lumen and engaging the distal segment of the inner element;
the occlusion device comprising a tubular scaffold extending from a proximal end to a distal end and defining a device lumen therethrough, the scaffold being formed from a plurality of interconnected and articulated members configured to self-expand from a closed configuration to an open configuration, a plurality of barbs extend from the articulated members with each barb, including an anchoring end, the anchoring end being disposed radially outward from the scaffold in the open configuration and adapted to embed into the cavity walls, a radially expandable inner matrix being disposed within the device lumen and attached to at least one of the articulated members the inner matrix being configured to promote body tissue growth within the body cavity; and
the occlusion device being coaxially arranged within the sheath lumen in the closed configuration such that the inner matrix is compressed within the device lumen, the occlusion device being deployable through the distal part of the outer sheath by relative axial movement of the outer sheath, and the scaffold, barbs, and extracellular matrix self-expand into the open configuration after deployment of the occlusion device.

15. The device of claim 14 wherein the anchoring end of the barbs are disposed substantially flush along the scaffold in the closed configuration within the outer sheath.

16. The device of claim 14 wherein the inner matrix further comprises at least one of an extracellular matrix, polyester, rayon, nylon, polytetrafluoroethylene, biocompatible polyurethanes, and mixtures thereof.

17. The assembly of claim 16 wherein the extracellular matrix further comprises small intestine submucosa.

18. The device of claim 14 wherein the scaffold and barbs are formed of a shape memory material including alloys of nickel-titanium.

19. A method of occluding a body cavity having body walls, the method comprising:
positioning an occlusion device within the body cavity to promote body tissue growth, the occlusion device comprising a tubular scaffold extending from a proximal end to a distal end and defining a device lumen therethrough, the scaffold being formed from a plurality of interconnected and articulated members configured to self-expand from a closed configuration to an open configuration, a plurality of barbs extend from the articulated members with each barb including an anchoring end, the anchoring end being disposed radially outward from the scaffold in the open configuration and adapted to embed into the cavity walls, a radially expandable inner matrix being disposed within the device lumen and attached to at least one of the articulated members is configured to promote body tissue growth within the body cavity;
expanding the occlusion device within the body cavity; and
attaching the anchoring ends of occlusion device to the body walls of the body cavity.

20. The method of claim 19 wherein the body cavity further comprises a patent foramen ovale.
